(19) United States
(12) Patent Application Publication
(10) Pub. No.: US 2022/0379314 A1
(43) Pub. Date: Dec. 1, 2022

(54) TEMPERATURE CONTROL APPARATUS FOR TEMPERATURE-CONTROLLED MATERIAL IN A CYLINDRICAL CONTAINER

(71) Applicant: Barkey GmbH & Co. KG, Leopoldshoehe (DE)

(72) Inventors: Armin Nowack, Guetersloh (DE); Christian Barkey, Bielefeld (DE)

(21) Appl. No.: 17/825,107

(22) Filed: May 26, 2022

(30) Foreign Application Priority Data
May 31, 2021 (DE) 10 2021 113 984.8

(51) Int. Cl. B01L 7/00; H05B 3/20

(52) U.S. Cl. CPC B01L 7/00; H05B 3/20; B01L 2300/1811

(57) ABSTRACT

An apparatus for temperature-controlling and thawing a temperature-controlled material which is arranged in at least one rigid container, including an openable housing containing a storage chamber for receiving the at least one container, and including a heating device containing a flat heating element which is operatively connected to the at least one container. A storage mat for receiving the at least one container is arranged between the flat heating element and the at least one container.

The invention relates to an apparatus for temperature-controlling and thawing a temperature-controlled material which is arranged in a rigid container, comprising an openable housing containing a storage chamber for receiving the container, comprising a heating device containing a flat heating element which is operatively connected to the container.

DE 10 2014 103 930 A1 discloses an apparatus for storing cylindrical containers, in particular test tubes, which has mutually spaced support strips with recesses such that a plurality of test tubes are held securely in the recesses one next to the other. The support strips consist of a rigid material.

DE 10 2018 112 194 A1 discloses an apparatus for temperature-controlling and thawing temperature-controlled material which is arranged in a container, said apparatus comprising a heating device for temperature-controlling a temperature-controlled material arranged in a storage chamber. The heating device has a flat heating element which, on both sides of the container that contains the temperature-controlled material and is designed as a bag, is in contact with said container and leads to uniform thawing of the temperature-controlled material by heat transfer. The size of the flat heating elements arranged on both sides of the temperature-controlled material is adapted to the dimensions of the bag containing the temperature-controlled material such that virtually the entire bag is surrounded by the heating elements. In addition, a mechanical actuating element is provided, by means of which the bag containing the temperature-controlled material is set in motion, preferably rhythmically, which accelerates the thawing process. However, if the temperature-controlled material is not contained in bags but in elongate rigid containers, e.g., test tubes, there is a risk that the test tubes will move away from their intended position between the heating elements during the thawing process, in particular to the edge of the storage chamber, so as to impair the thawing result.

The object of the present invention is therefore to further develop an apparatus for temperature-controlling and thawing a temperature-controlled material arranged in a rigid container in such a way as to ensure uniform thawing or temperature-control of the temperature-controlled material contained in the container.

To achieve this object, the invention is characterized, together with the preamble of claim 1, in that a storage mat for receiving the container is arranged between the flat heating element and the container.

The particular advantage of the invention is that storing the rigid containers on a storage mat ensures that they are stored in a precise position and prevents undesirable slipping. Advantageously, this allows controlled, uniform thawing of the temperature-controlled material contained in the containers.

According to a preferred further development of the invention, the storage mat consists of a flexible material such that said storage mat can follow or compensate for the change in position of the containers caused by a movement of the flat heating element during the thawing and/or temperature control process. In addition, the flexibility of the storage mat can mitigate the impact process acting on the containers.

According to a further development of the invention, the storage mat is formed of a silicone material, preferably a silicone rubber material. Advantageously, this allows the storage mat to adapt optimally to the storage environment.

According to a further development of the invention, the containers are each cylindrical. The storage mat has a plurality of elongate receptacles having openings such that the heating element arranged on a rear side of the storage mat can come into direct contact with the container. Heat transfer can thus be improved.

According to a further development of the invention, the storage mat and/or the heating element is associated with a mechanically movable actuating element, by means of which the heating element and/or the storage mat can be made to move back and forth periodically and/or aperiodically. Advantageously, the storage mat can follow this back-and-forth movement and ensure secure storage of the containers during the thawing/temperature control process.

According to a further development of the invention, the mechanically movable actuating element is rigidly formed having a plurality of thin struts, which are preferably formed into a rigid frame. By pivoting the thus formed rigid frame back and forth, a movement of the containers that promotes temperature control or thawing can be brought about independently of the heating method so as to accelerate the temperature control or thawing of the contents of the containers.

According to a further development of the invention, a number of retaining tabs protrude from the outside of the storage mat; said retaining tabs can be supported, for example, on an upright side wall of the storage chamber if the dimensions of the storage mat are larger than a base area of the storage chamber or supporting surface of the housing. Alternatively or additionally, the retaining tabs can also be used to partially cover an accessory, which is used for processing the containers. In this case, the dimensions of the storage mat are smaller than or equal to the base area of the storage chamber.

Further advantages of the invention are apparent from the further dependent claims.

Exemplary embodiments of the invention are explained in more detail below with reference to the drawings.

The drawings show:

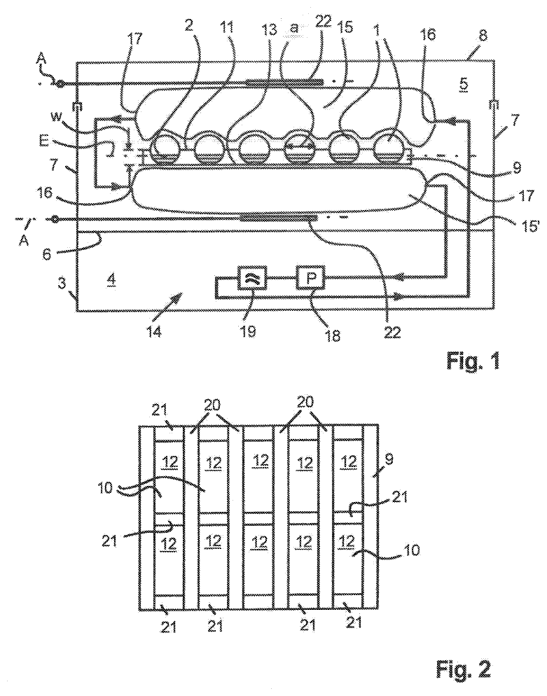

FIG. 1 a schematic cross section through a storage chamber of an apparatus according to the invention,

FIG. 2 a plan view of a storage mat that is not provided with containers,

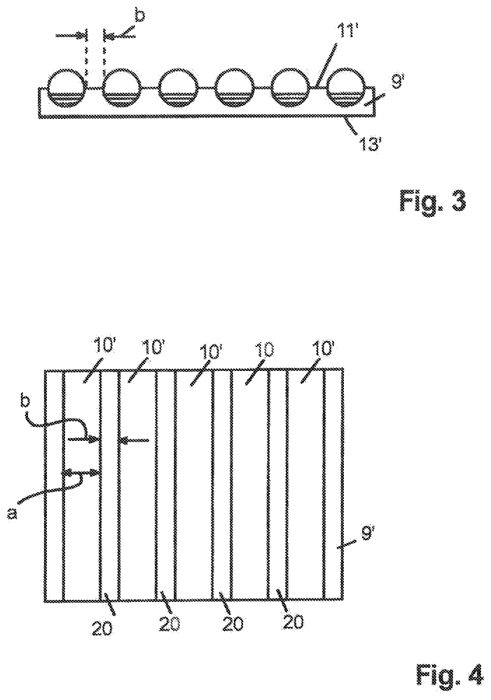

FIG. 3 a cross section through an alternative storage mat together with containers stored thereon, and

FIG. 4 a plan view of the storage mat according to FIG. 3 without containers stored thereon,

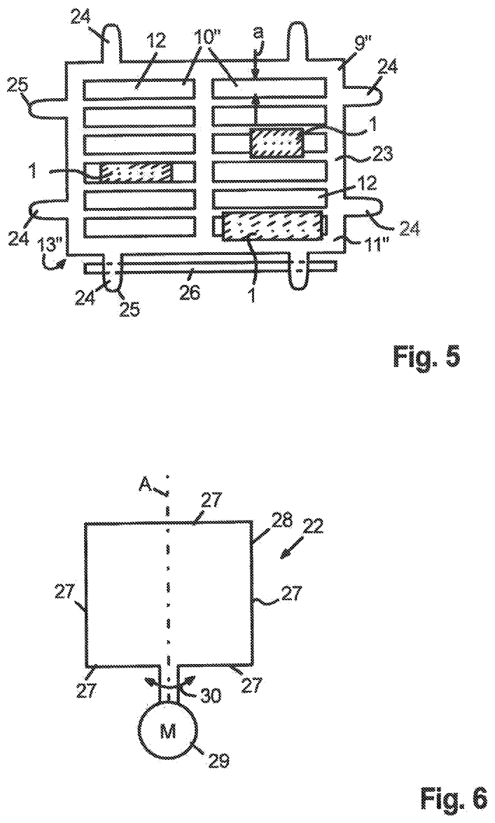

FIG. 5 a plan view of a storage mat according to a further embodiment, and

FIG. 6 a plan view of a rigid actuating element, by means of which the storage mat and/or the heating element can be moved back and forth.

An apparatus according to the invention for temperature-controlling and/or thawing a temperature-controlled material 2 arranged in elongate rigid containers 1 has a housing 3 which has a base chamber 4 in a lower region and a storage chamber 5 in an upper region. The containers can be designed, for example, as vials (small bottles) for samples or the like.

The storage chamber 5 is delimited at the bottom, and the base chamber 4 at the top, by a rigid partition 6. The storage chamber 5 is pot-shaped and has upright side walls 7 which define an upper opening. The upper opening in the storage chamber 5 can be opened and closed by means of a hinged cover 8.

A storage mat 9 for the positionally precise storage or holding of a plurality of elongate containers 1, preferably cylindrical containers, in particular test tubes, is provided in the storage chamber 5. For this purpose, the storage mat 9 has elongate receptacles 10 such that the cylindrical containers 1 can be placed on the storage mat 9 from an upper side thereof. The elongate receptacles 10 are preferably designed as elongate recesses such that the containers 1 are arranged recessed at least in regions in relation to an upper side 11 of the storage mat 9. According to a first embodiment of the storage mat 9 according to FIGS. 1 and 2, the elongate recesses 10 have openings 12, preferably elongate openings 12, in regions, such that the containers 1 can, in the storage position, be arranged at least flush with a lower side 13 of the storage mat 9, as shown in FIG. 1, or project beyond a plane of the lower side 13.

The apparatus has a heating device 14 which comprises two flat heating elements 15, 15′. An upper heating element 15 is arranged above the storage mat 9 provided with the containers 1, and a lower heating element 15′ is arranged below the storage mat 9 provided with the containers 1. The two heating elements 15, 15′ are each designed as a plastic bag having an inlet 16 and an outlet 17 such that a temperature-controlled medium, preferably a medium in a liquid physical state, can flow through the heating elements 15, 15′. For this purpose, the heating device 14 has a pump 18 and a heat source 19, which are preferably arranged in the base chamber 4. The pump 18 and the heat source 19 are connected to the heating elements 15, 15′ via hose lines. Preferably, the heating elements 15, 15′ are connected in series in the direction of flow.

As can be seen from FIG. 2, projected onto a plane E of extension of the storage mat 9, the openings 12 span an area greater than half of the area of the elongate receptacle 10, similarly projected onto the plane E of extension. Preferably, the area of the openings 12 is in a range between 50% and 90% of the projected area of the elongate receptacle 10 such that the largest possible area of containers 1 is in direct contact with the lower heating element 15′ on a side facing the lower heating elements 15′. Direct contact of the containers 1 with the upper heating element 15 and the lower heating element 15′ is thus achieved, which improves the introduction of heat into the containers 1 during thawing thereof.

In the first exemplary embodiment according to FIGS. 1 and 2, a transverse extension q of the containers 1 is greater than a wall thickness w of the storage mat 9 in an intermediate region 20 adjacent to the elongate receptacles 10. In the intermediate region 20, in a flat initial position of the storage mat 9, the upper side 11 thereof extends in parallel with the lower side 13. The elongate receptacles 10 arranged in a receiving region of the storage mat 9 have a number of receiving portions 21 that are arranged axially offset from one another and are adapted to the outer contour of the containers 1. The containers 1 rest on the receiving portions 21 of the receptacles 10. If the containers 1 are cylindrical in cross section, the receiving portions 21 are circular in cross section.

In the present exemplary embodiment, the storage mat 9 consists of a flexible material, preferably a silicone material, in particular a silicone rubber material.

In addition, a mechanically movable actuating element 22 can be arranged in the storage chamber 5, by means of which the upper heating element 15 and/or the lower heating element 15′ can be set in motion. The mechanical actuating element 22 can be designed, for example, as a paddle element which can be moved back and forth periodically and/or aperiodically about an axis A, specifically in a predetermined acute or obtuse angle range. The axis A of rotation extends in parallel with the plane E of extension of the storage mat 9 or with a plane of extension of the heating elements 15, 15′.

A control device (not shown) for the actuating element 22 is arranged in the base chamber 4. This control device can also be used to control the pump 18 and the heat source 19 if the heat source 19 is designed as an electrically operated heat source, for example a resistance heater.

In the present exemplary embodiment, the heating elements 15, 15′ are each associated with an actuating element 22, the actuating element 22 being arranged on a side of the heating elements 15, 15′ facing away from the storage mat 9.

According to an alternative embodiment of the invention (not shown), just a single actuating element 22 can also be arranged in the storage chamber 5, said actuating element acting on the heating elements 15, 15′ on a side thereof facing away from the storage mat 9 or on a side thereof facing the storage mat 9.

According to an alternative embodiment, the actuating element 22 can be arranged directly at one end of the upper heating element 15 and/or the lower heating element 15′, with the back-and-forth movement being upward and downward. This allows the end of the heating element 15, 15′ to be moved up and down periodically and/or aperiodically such that a wave motion continues from this point to an opposite end of the heating element 15, 15′. The actuating element 22 can be rigidly connected to the heating element 15 or 15′.

According to an alternative embodiment of the invention according to FIGS. 3 and 4, a storage mat 9′ is provided, which, unlike the storage mat 9, does not have any openings. A lower side 13′ of the storage mat 9′ is flat. Only an upper side 11′ of the storage mat 9′ has continuous elongate recesses 10′ in which the containers 1 are stored.

Identical components or component functions are provided with the same reference numbers. Between the elongate recesses 10′, the storage mat 9′ has intermediate regions 20 in which the upper side 11′ and the lower side 13′ of the storage mat 9, in a flat position, extend in parallel with one another.

In this embodiment, the lower side 13′ of the storage mat 9′ is in full contact with the lower heating element 15′. Since the storage mat 9′ consists of a flexible and heat-conducting material, the heat emitted by the lower heating element 15′ can be transferred to the containers 1.

According to an alternative embodiment of the invention (not shown), the distance b between adjacent elongate receptacles 10, 10′ can be made smaller than a width a of the recess 10, 10′. To increase the packing density of the containers 1 on the storage mat 9, 9′, the width b of the intermediate region 20 can be selected to be relatively small, for example in the millimeter range.

For example, the temperature-controlled material 2 can be in the form of a liquid or solid gene or stem cell material.

According to an alternative embodiment of the invention, the heating elements 15, 15′ can be designed as temperature control pads or gel pads that are heated electrically in both cases.

According to a further embodiment of a storage mat 9″ according to FIG. 5, said storage mat has a peripheral flat frame 23 which surrounds a plurality of rectangular recesses 10″. The flat frame 23 has a width in the range of 250 mm to 300 mm. The elongate recesses 10″ are formed as openings 12 and each have a width a in the range of 10 mm to 50 mm. The widths a of the recesses 10″ are preferably equal.

In the present embodiment, two rows of six recesses 10″ each are arranged offset from one another. The containers 1 to be stored are placed on an upper side 11″ of the storage mat 9″. A number of retaining tabs 24 are distributed on an outer side of the closed peripheral flat frame 23, the free end 25 of said retaining tabs projecting substantially perpendicularly outwardly from the flat frame 23. The retaining tabs 24 can be used to support the storage mat 9″ or the containers 1 on an edge of the housing 3 and/or on the lower heating element 15. Alternatively or additionally, the retaining tabs 24 can also be used to partially cover an accessory 26 placed at the edge of the storage chamber 5.

The storage mat 9″ is made of a flexible material, preferably a silicone material.

A wall thickness w of the storage mat 9″ is in a range from 1 mm to 5 mm, preferably 2 mm. The recesses 10″ have dimensions of 125 mm×25 mm.

FIG. 6 shows the dimensions of the mechanically movable actuating element 22. In contrast to the storage mat 9″, the actuating element 22 is rigid and has a plurality of thin, perpendicularly interconnected struts 27. The struts 27 can be rectangular or circular in cross section. Preferably, the struts 27 are made of a solid material, preferably a solid plastic material. The struts 27 are straight.

The struts 27 form a rigid frame 28, which preferably has smaller dimensions than the flat frame 23 of the storage mat 9″. Alternatively or additionally, the struts 27 can also be curved such that the frame 28 is circular or elliptical in the plane of extension thereof.

The rigid frame 28 or the struts 27 are moved back and forth periodically and/or aperiodically through a pivot angle 30 by means of an actuating element 29, which can be designed, for example, as an electric servomotor, with a pivot axis or axis A of rotation forming an axis of symmetry of the rigid frame 28. The axis A of rotation extends in parallel with or in the plane E of extension of the storage mat 9″.

The actuating element 22 can be arranged—as shown in FIG. 1—on a side of the lower heating element 15′ facing away from the storage mat 9″ and/or on a side of the upper heating element 15 facing away from the storage mat 9″. Alternatively or additionally, the actuating element 22 can also be arranged between the upper heating element 15 and the upper side 11″ of the storage mat 9″ and/or between the lower heating element 15′ and a lower side 13″ of the storage mat 9″.

1. An apparatus for temperature-controlling and thawing a temperature-controlled material (2), which is arranged in at least one rigid container (1), comprising an openable housing (3) containing a storage chamber (5) for receiving the at least one container (1), comprising a heating device (14) containing a flat heating element (15, 15′) which is operatively connected to the at least one container (1), wherein a storage mat (9, 9′, 9″) for receiving the at least one container (1) is arranged between the flat heating element (15, 15′) and the at least one container (1).

2. The apparatus according to claim 1, wherein the storage mat (9, 9′, 9″) consists of a flexible material.

3. The apparatus according to claim 1, wherein the at least one container (1) is cylindrical and wherein that the storage mat (9, 9′, 9″) has a plurality of elongate receptacles (10, 10′, 10″) for respectively receiving a plurality of the at least one containers (1).

4. The apparatus according to claim 3, wherein the elongate receptacles (10, 10′, 10″) are each formed as elongate recesses.

5. The apparatus according to claim 1, wherein the flat heating element (15, 15′) is a plastic bag containing a temperature-controlled medium

6. The apparatus according to claim 5, wherein the plastic bag is operatively connected to a pump (18) arranged outside the storage chamber (5) and to a heat source (19) in order to temperature-control the temperature-controlled medium to a predetermined temperature.

7. The apparatus according to claim 5, wherein the temperature-controlled medium is in a liquid physical state.

8. The apparatus according to claim 3, wherein the storage mat (9) and the plurality of containers (1) stored thereon are arranged between two flat heating elements (15, 15′).

9. The apparatus according to claim 3, wherein the elongate recesses (10, 10′, 10″) in the storage mat (9) have at least one opening (12).

10. The apparatus according to claim 3, wherein the recesses (10, 10′, 10″) in the storage mat (9, 9′, 9″) have a width (a) in the range of 10 mm to 50 mm.

11. The apparatus according to claim 1, wherein the storage mat (9, 9′, 9″) has a number of retaining tabs (24) which can be pivoted out of a plane (E) of extension of said storage mat and which are for partially covering the container (1) and/or an accessory (26).

12. The apparatus according to claim 3, wherein the storage mat (9, 9′, 9″) has a number of retaining tabs (24) which can be pivoted out of a plane (E) of extension of said storage mat and which are for partially covering the container (1) and/or an accessory (26), wherein the storage mat (9, 9′, 9″) has a peripheral flat frame (23) which surrounds the plurality of recesses (10″) and on the outside of which the plurality of retaining tabs (24) are arranged.

13. The apparatus according to claim 12, wherein a free end (25) of the retaining tab (24) adjoins perpendicularly from the flat frame (23) of the storage mat (9″).

14. The apparatus according to claim 1, wherein the storage mat (9, 9′) and/or the heating element (15, 15′) is associated with a mechanically movable actuating element (22) in such a way that, by periodic and/or aperiodic movement of the actuating element (22), the storage mat (9, 9′) can be made to move back and forth transversely to a plane (E) of extension of said storage mat.

15. The apparatus according to claim 14, wherein the mechanically movable actuating element (22) is rigid and comprises a plurality of interconnected straight and/or curved struts (27) to form a rigid frame (28) having smaller dimensions than the flat frame (23) of the storage mat (9″).

16. The apparatus according to claim 14, wherein the actuating element (22) is arranged between an upper heating element (15) and an upper side (11″) of the storage mat (9, 9′, 9″) or on a side of the upper heating element (15) facing away from the storage mat (9, 9′, 9″) and/or between a lower heating element (15′) and a lower side (13′) of the storage mat (9″) or on a side of the lower heating element (15′) facing away from the storage mat (9″), the actuating element (22) being controllable about a pivot axis (A) which extends in parallel with the plane (E) of extension of the storage mat (9″).

17. The apparatus according to claim 1, wherein the storage mat (9, 9′, 9″) consists of a one-piece silicone material.
